(19) United States
(12) Patent Application Publication
(10) Pub. No.: US 2015/0282819 A1
(43) Pub. Date: Oct. 8, 2015

(54) ONE PIECE HANDHELD STAPLE HOLDER AND DRILL GUIDE FOR APPLICATION OF A MEDICAL SHAPE MEMORY, ELASTIC OR SUPERELASTIC NITINOL MEMORY BONE STAPLE

(71) Applicant: Neutin Orthopedics, LLC, Millersville, MD (US)

(72) Inventors: Albert Austin, Millersville, MD (US); Steven Keith Neufeld, Washington, DC (US)

(21) Appl. No.: 14/678,854

(22) Filed: Apr. 3, 2015

Related U.S. Application Data
(60) Provisional application No. 61/995,172, filed on Apr. 3, 2014.

(51) Int. Cl. A61B 17/17

(52) U.S. Cl. CPC A61B 17/17; A61B 2017/0023

(57) ABSTRACT

A disposable or re-usable handheld, one piece device is provided for pre-drilling and application of a shape memory or elastic staple for fixation in bone surgery. Various embodiments include a one piece device that has set holes at one end to allow for pre-drilling for the shape memory or elastic staple legs and includes an application feature at the other end to disengage the shape memory or elastic staple from the one piece device. The one piece device can come in different sizes depending on the size of the shape memory or elastic staple. The one piece handheld shape memory or elastic staple holder and drill guide allows for a handheld device to accomplish both pre-drilling and application of the shape memory or elastic staple for surgical bone fixation procedures.

### CROSS-REFERENCE TO RELATED APPLICATIONS

The present application claims the benefit of U.S. Provisional Application No. 61/955,172, filed Apr. 3, 2014, which is hereby incorporated by reference herein in its entirety.

### FIELD

This present disclosure relates generally to medical devices, and, more particularly, to medical devices for application of medical staples such as medical shape memory and/or elastic/superelastic memory bone staples.

### BACKGROUND

Bone fixation staples have been used in medical surgery of the foot and hand for many years, evolving from rigid stainless steel, titanium or cobalt-chrome U-shaped staples that could be manipulated to compress two adjacent bone segments to shape memory and elastic bone fixation nitinol staples. Nitinol is a nickel-titanium alloy that was discovered in 1958 by metallurgist William J. Buehler working at the U.S. Naval Ordnance Laboratory. Shape memory and elastic bone fixation nitinol staples have been in clinical use for many years and have been used in orthopedics and podiatry foot surgery for a variety of surgical fixation methods. Nitinol is a composition of both Nickel and Titanium which is called a shape memory device when forged in the shape of a medical staple. A shape memory alloy is an alloy that remembers its original, cold forged shape and can return to its original shape (i.e., original memory position or pre-determined shape) when heated by a separate activator or under body temperature. This nitinol composition gives the shape memory and elastic bone staples their memory and compression qualities. Shape memory and elastic alloy bone staples provide the advantage of a sustained compressive force across the bone fixation site.

Elastic or superelastic memory staples have the same memory compression characteristics as shape memory but without the effect of heat or cold. They are elastic and spring back to their compressed position when released from a holder that holds the elastic staple legs straight.

Once released, the staple legs of the elastic memory nitinol staple bend inwards and compress to their original memory position. The surgeon pre-drills holes for the shape memory and/or elastic bone fixation staple legs to be inserted into the bones for placement of the staple.

There have been various methods of introducing the shape memory and/or elastic staple to the bone surface such as: using a hemostat clamp, surgical clamp, staple forceps, a special plastic staple actuator or holder to push the staple into the pre-drilled holes for placement. There have been various methods of drilling the holes to implant a shape memory or elastic staple such as: adjustable drill guides positioned exactly according to the staple width/size desired, manual drilling or fixed drill guides that are fixed to the precise size of the desired staple. These drill guides have come in both disposable and re-usable materials, from various metals to medical grade plastic materials.

The surgical implantation procedure, for shape memory and/or elastic alloy staples and medical bone staples, are almost identical in the technique to implant them into the bone. The surgeon decides what size (e.g., width, length, and diameter legs) staple is required. The surgeon then selects the proper drill guide for that staple which is separate from the staple, either in a reusable instrument tray or disposable kit tray. The surgeon then selects the appropriate drill bit device to pre-drill the holes in the bone for the staple legs which is normally a re-usable or disposable medical grade drill bit that closely or exactly matches the diameter of the staple legs. The surgeon places the proper drill guide on the bone surface where the staple will be implanted. The surgeon drills the appropriate holes for the staple. The surgeon, with a separate device to hold the staple, then introduces the staple into the pre-drilled holes. This separate device holds the implantable shape memory and/or elastic staple to be implanted into the predrill holes. The surgeon disengages, pushes, or removes the staple from the staple holder or actuator and positions the staple into the pre-drilled holes for final placement of the shape memory or elastic staple into the bone.

The use of a separate drill guide and a separate holding device can be inefficient when time is spent verifying that that the drill guide size matches the desired staple size and/or that the holding device size matches the desired staple size. The use of separate devices can also lead to the drilling of holes that are not the correct size if a drill guide is used that is not the right size for the desired staple.

### SUMMARY

In one or more embodiments, a disposable or re-usable one piece handheld staple holder and drill guide device for application of a medical shape memory, elastic or superelastic nitinol memory bone staple comprises a drill guide section, a middle portion, and a grooved section. The drill guide section can be for pre-drilling of the holes for the shape memory or elastic medical bone staple to be inserted into the bone. The middle portion can allow the user to hold the one piece handheld shape memory staple holder and drill guide. The grooved section can hold the shape memory or elastic medical bone staple.

In one or more embodiments, a one piece combination staple holder and drill guide medical device for application of a medical staple comprises a drill guide portion, a staple holder portion, and a middle portion. The drill guide portion can comprise two drill holes arranged a distance apart, the distance being based on a size of the medical staple. The staple holder portion can comprise a groove, the medical staple being insertable into the groove such that, when inserted, legs of the medical staple remain in a fixed position. The middle portion can be coupled to the drill guide portion and the staple holder portion.

In one or more embodiments, a medical device comprises a staple holder portion and a drill guide portion. The staple holder portion can be configured such that a medical staple can be releasably attachable to the medical device at the staple holder portion such that, when attached, legs of the medical staple remain in a fixed position. The drill guide portion can comprise two drill holes being a distance apart, the distance being based on a size of the medical staple with the legs at the fixed position. The diameter of the two drill holes can be based on the diameter of the legs of the medical staple.

Objects and advantages of embodiments of the disclosed subject matter will become apparent from the following description when considered in conjunction with the accompanying drawings.

### BRIEF DESCRIPTION OF THE DRAWINGS

Embodiments will hereinafter be described with reference to the accompanying drawings, which have not necessarily been drawn to scale. Where applicable, some features may not be illustrated to assist in the illustration and description of underlying features. Throughout the figures, like reference numerals denote like elements. As used herein, various embodiments can mean one, some, or all embodiments.

FIG. 1 is a top/side oblique view of a one piece handheld shape memory staple holder and drill guide, according to one or more embodiments of the disclosed subject matter.

FIG. 2 is a top view of the one piece handheld shape memory staple holder and drill guide of FIG. 1, according to one or more embodiments of the disclosed subject matter.

FIG. 3 is a side view of the one piece handheld shape memory staple holder and drill guide of FIG. 1, according to one or more embodiments of the disclosed subject matter.

FIG. 4 is a cross sectional view of the one piece handheld shape memory staple holder and drill guide of FIG. 1, according to one or more embodiments of the disclosed subject matter.

FIG. 5 is an oblique view of the one piece handheld shape memory staple holder and drill guide of FIG. 1, according to one or more embodiments of the disclosed subject matter.

### DETAILED DESCRIPTION

In one or more embodiments of the present disclosure, a disposable or re-usable handheld, one piece device is provided for pre-drilling and application of a shape memory or elastic staple for fixation in bone surgery. One or more embodiments include a one piece device that has set holes at one end to allow for pre-drilling for the shape memory or elastic staple legs and include an application feature at the other end to disengage the shape memory or elastic staple from the one piece device. Embodiments can come in different sizes depending on the size of the shape memory or elastic staple. The one piece handheld shape memory or elastic staple holder and drill guide allows for a handheld device to accomplish both the pre-drilling of holes for and application of the shape memory or elastic staple for surgical bone fixation procedures.

In one or more embodiments of the present disclosure, a one piece handheld device holder for pre-drilling, holding, and/or application (e.g., insertion) of shape memory and/or elastic medical staples is provided for use in bone surgery. In various embodiments, a one piece handheld shape memory staple holder and drill guide for applications of shape memory and/or elastic memory bone staples allows for pre-drilling of the staple holes and application of the staple into the pre-drilled holes. The present disclosure overcomes deficiencies in both the one piece bone fixation staple holders and the separate one piece bone fixation staple drill guides used to implant bone fixation staples.

In one or more embodiments, the one piece handheld shape memory staple holder and drill guide is specifically configured for holding the shape memory staple and/or elastic staple for implantation, for providing a specific drilling guide for that specific staple, all in one piece. The one piece handheld shape memory staple holder and drill guide can be made out of re-usable or disposable medical grade material (e.g., medical grade metal, plastic, wood, etc.). The one piece handheld shape memory staple holder and drill guide can come in a variety of sizes depending on the size of the shape memory and/or elastic staple that it is holding before application.

An advantage of embodiments of the present disclosure is that the embodiments provide for handheld pre-drilling for the shape memory and/or elastic bone fixation staple, holding the staple, and application (e.g. insertion) of the shape memory and/or elastic bone fixation staple, all in a one piece device.

Another advantage of the disclosed embodiments is that the one piece device can come in a variety of sizes depending on the shape memory and/or elastic bone fixation staple size desired and held in the one piece device. Various sizes allow pre-drilling and application using a one piece handheld shape memory staple holder and drill guide device for a variety of shape memory and/or elastic bone fixation staples desired for the particular application to the anatomy of the bone location.

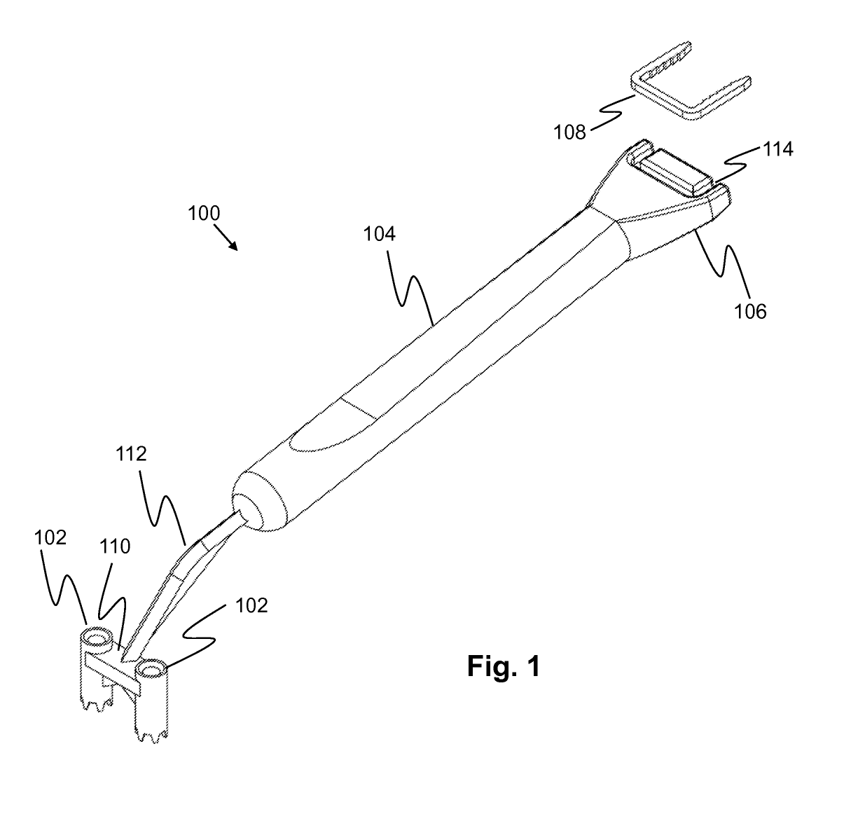

FIG. 1 is a top/side oblique view of a one piece handheld shape memory staple holder and drill guide 100, according to one or more embodiments of the disclosed subject matter. One piece handheld shape memory staple holder and drill guide 100 is configured for application of a shape memory and/or elastic medical staple 108 used for bone fixation.

The one piece device 100 includes a drill guide 110 with two aligned drilling holes 102 that are aligned based on the particular width/size (e.g., width, length, and/or diameter) of a bone fixation staple 108 (e.g., used to fixate the bone), a middle section 104 that allows a user to hold the one piece device 100, and a grooved staple holder 106 that holds the staple 108 in place before application and insertion into the bone. One piece device 100 can include a portion 112 between middle section 104 and drill guide 110.

One piece device 100 provides the user with a one piece handheld shape memory staple holder and drill guide for application of a shape memory or elastic medical bone staple 108. One piece handheld shape memory staple holder and drill guide 100 can be provided in various sizes depending on the size/width of the shape memory or elastic bone staple 108. For example, the drill guide section 110 can be provided in different widths (i.e., different distances between the drilling holes 102) and diameters of the drilling holes 102 depending on the size of the shape memory or elastic bone staple 108 that is held (or can be held) by the grooved staple holder portion 106.

In some embodiments, one piece device 100 is made of reusable medical grade material. In some embodiments, one piece device 100 is made of disposable medical grade material. One piece handheld shape memory staple holder and drill guide 100 can be made of one or more of medical grade plastic material, medical grade plastic like material, medical grade metal alloy material, and/or any other suitable material that can be molded or manufactured.

In various embodiments, one piece device 100 can be made in various thicknesses or widths depending on characteristics (e.g. size/type) of the staple with which one piece device 100 is configured to be used (e.g., the shape memory or elastic bone staple 108 used and held by the one piece handheld shape memory staple holder and drill guide 100).

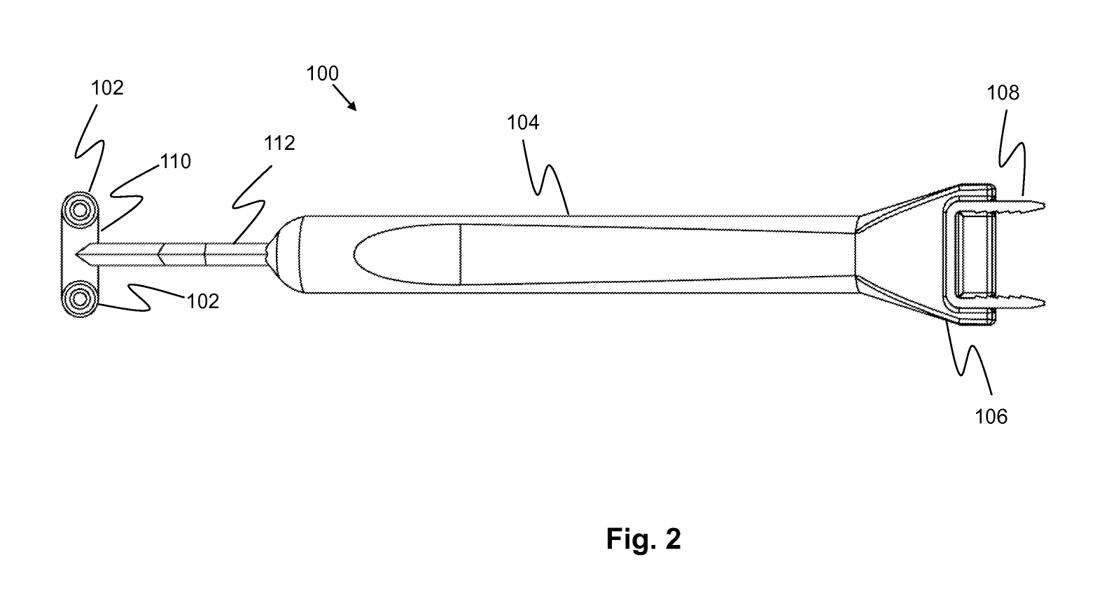

FIG. 2 is a top view of the one piece handheld shape memory staple holder and drill guide 100 of FIG. 1, according to one or more embodiments of the disclosed subject matter. FIG. 2 illustrates how staple 108 can be held by groove 114 of staple holder portion 106.

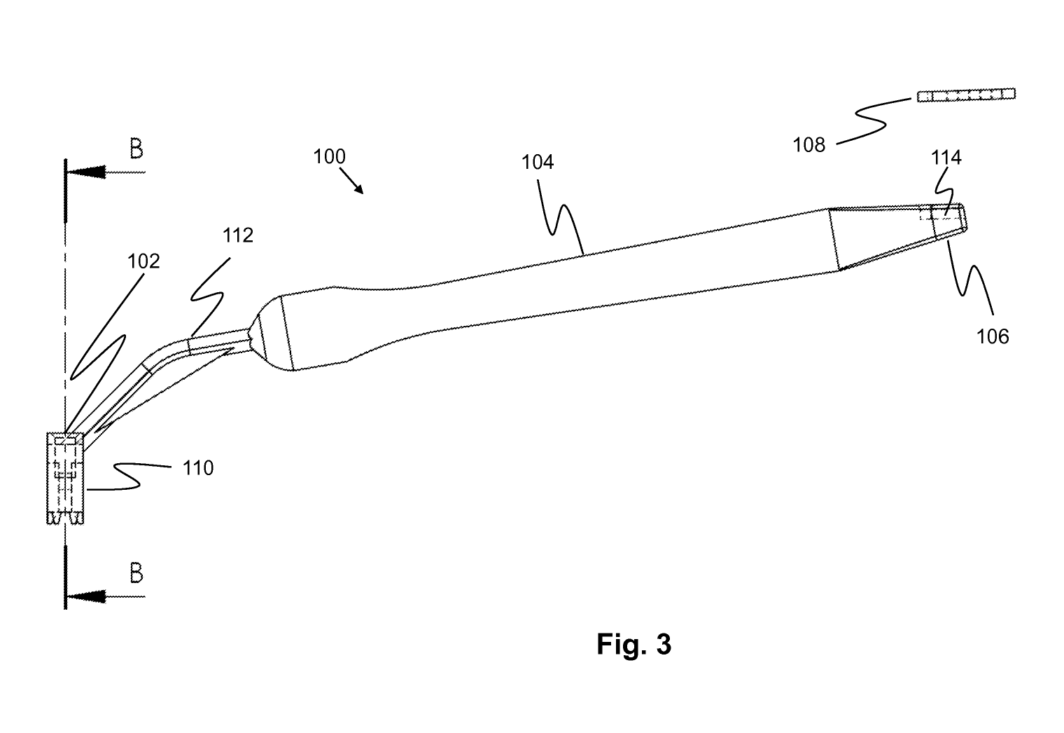

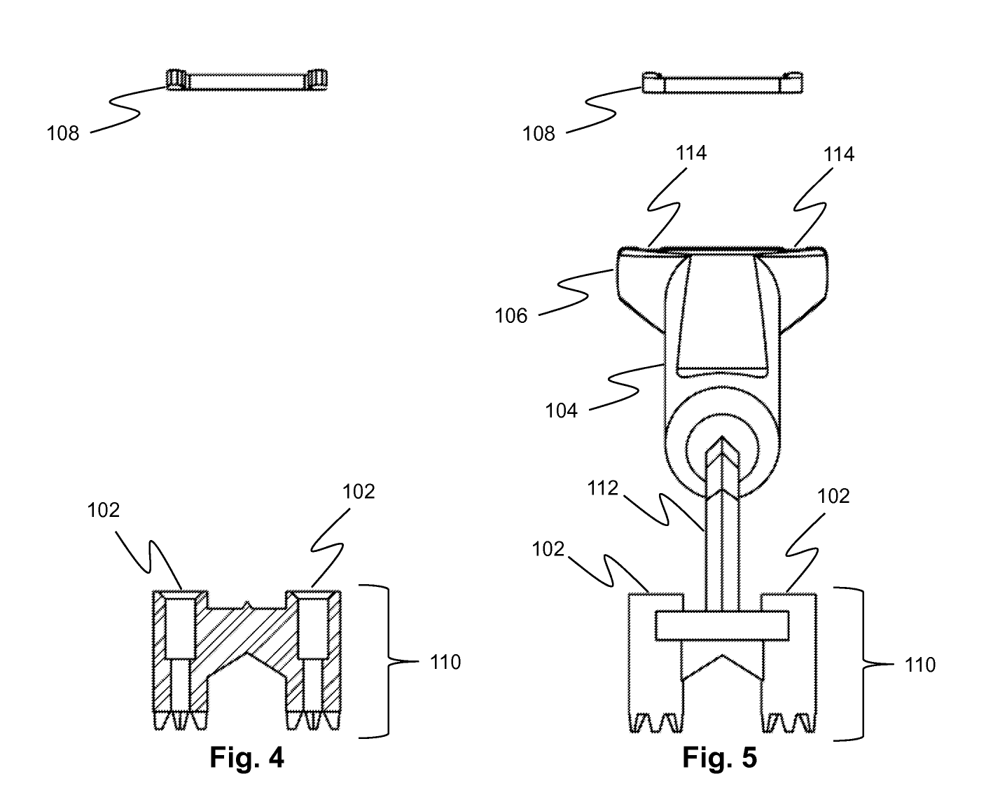

FIG. 3 is a side view of the one piece handheld shape memory staple holder and drill guide 100 of FIG. 1, according to one or more embodiments of the disclosed subject matter. A cross sectional view at plane B of one piece handheld shape memory staple holder and drill guide 100 is shown in FIG. 4.

FIG. 4 is a cross sectional view of the one piece handheld shape memory staple holder and drill guide 100 of FIG. 1, according to one or more embodiments of the disclosed subject matter. FIG. 4 shows a cross sectional view of drill guide 110 at plane B as shown in FIG. 3. The diameter of drill holes 102 can be based on the size of the legs of staple 108 so that drill holes 102 can be used to drill holes into bone such that the legs of staple 108 can be inserted into the drilled holes.

FIG. 5 is an oblique view of the one piece handheld shape memory staple holder and drill guide 100 of FIG. 1, according to one or more embodiments of the disclosed subject matter. FIG. 5 provides an oblique view of one piece device 100 from the drill guide side of the device 100.

It will be appreciated that although one piece device 100 has been shown as a device for use with a staple having two legs, in some embodiments one piece devices can be adapted to hold staples having more than two legs. In some such embodiments, the one piece devices can include a drill guide having a drill hole for each of the legs and a staple holder adapted to hold the staple for application into the bone.

In some embodiments, a method comprises holding a unitary device in a first position to drill holes into bone, the unitary device having a drill guide and an attachment tool. The method can include holding the same unitary device in a second position to introduce a sample into the holes, the staple being held by the attachment tool of the unitary device. In some embodiments, the method can include disengaging the staple from unitary device and positioning the staple into the holes for final placement of the staple into the bone. The attachment tool can hold the staple such that legs of the staple remain in a fixed position in which the legs can be inserted into the holes drilled using the drill guide. In some embodiments, holding the same unitary device in another position to introduce a sample into the holes includes rotating the unitary device. In some embodiments, the staple can be attached to the unitary device prior to holding the unitary device in the first position to drill holes into bone. The drill guide and the attachment tool of the unitary device can both be adapted for use with the same particular size or type of staple, such as, for example, a particular size of medical shape memory and/or elastic/superelastic nitinol memory bone staple, such that a staple attachable to the attachment tool for holding can be inserted into holes drilled using the drill guide of the unitary device. In some embodiments, the drill guide can include drill holes, the drill holes can have diameters based on the diameters of the legs of the staple, and the drill holes can be arranged to match the arrangement of the legs of the staple such that the staple can be inserted into holes drilled using the drill holes.

In some embodiments the unitary device can be disposable. In some embodiments the method can include disposing the unitary device. In some embodiments the unitary device can be reusable. In some embodiments, the unitary device can compose medical grade plastic material, medical grade plastic like material, and/or medical grade metal alloy material.

In some embodiments, the unitary device can be one piece device 100 shown in FIGS. 1-5, and the drill guide and attachment tool can be drill guide 110 and staple holder 106, respectively.

Features of the disclosed embodiments may be combined, rearranged, omitted, etc., within the scope of the invention to produce additional embodiments. Furthermore, certain features may sometimes be used to advantage without a corresponding use of other features.

It is, thus, apparent that there is provided, in accordance with the present disclosure, a one piece handheld staple holder and drill guide for application of a medical shape memory and/or elastic/superelastic nitinol memory bone staple. Many alternatives, modifications, and variations are enabled by the present disclosure. While specific embodiments have been shown and described in detail to illustrate the application of the principles of the invention, it will be understood that the invention may be embodied otherwise without departing from such principles. Accordingly, Applicants intend to embrace all such alternatives, modifications, equivalents, and variations that are within the spirit and scope of the present invention.

1. A disposable or re-usable one piece handheld staple holder and drill guide device for application of a medical shape memory, elastic or superelastic nitinol memory bone staple, the device comprising:
a drill guide section for pre-drilling of the holes for the shape memory or elastic medical bone staple to be inserted into the bone;
a middle portion allowing the user to hold the one piece handheld shape memory staple holder and drill guide; and
a grooved section to hold the shape memory or elastic medical bone staple.

2. The device of claim 1, wherein the drill guide section comprises two drill holes arranged a distance apart, the distance being based on a size of the staple.

3. The device of claim 1, wherein the grooved section comprises a groove, the staple being insertable into the groove such that, when inserted, legs of the staple remain in a fixed position.

4. The device of claim 1, wherein a staple is releasably attachable to the device at the grooved section such that, when attached, legs of the staple remain in a fixed position.

5. The device of claim 4,
wherein the drill guide section comprises two drill holes being a distance apart, the distance being based on a size of the staple with the legs at the fixed position, and
wherein the diameter of the two drill holes is based on the diameter of the legs of the medical staple.

6. The device of claim 1, wherein the drill guide section is coupled to one end of the middle portion, and the grooved section is coupled to another end of the middle portion.

7. The device of claim 1, wherein the device is composed of: medical grade plastic material; medical grade plastic like material, and/or medical grade metal alloy material.

8. A one piece combination staple holder and drill guide medical device for application of a medical staple, the medical device comprising:
a drill guide portion comprising two drill holes arranged a distance apart, the distance being based on a size of the medical staple;
a staple holder portion comprising a groove, the medical staple being insertable into the groove such that, when inserted, legs of the medical staple remain in a fixed position; and
a middle portion coupled to the drill guide portion and the staple holder portion.

9. The medical device of claim 8, wherein the two drill holes are arranged a distance apart based on the size of the medical staple.

10. The medical device of claim 6, wherein the drill guide portion is coupled to one end of the middle portion, and the staple holder portion is coupled to another end of the middle portion.

11. The medical device of claim 8, wherein the medical staple is a medical shape memory and/or elastic/superelastic memory staple.

12. The medical device of claim 11, wherein the medical staple is a nitinol staple.

13. The medical device of claim 8, wherein the medical device is reusable.

14. The medical device of claim 8, wherein the medical device is disposable.

15. A medical device comprising:
a staple holder portion, a medical staple being releasably attachable to the medical device at the staple holder portion such that, when attached, legs of the medical staple remain in a fixed position;
a drill guide portion comprising two drill holes being a distance apart, the distance being based on a size of the medical staple with the legs at the fixed position; and
the diameter of the two drill holes being based on the diameter of the legs of the medical staple.

16. The medical device of claim 15, the medical device further comprising:
a middle portion;
wherein the drill guide portion is coupled to one end of a middle portion, and the staple holder portion is coupled to another end of the middle portion.

17. The medical device of claim 15, wherein the medical staple is a medical shape memory and/or elastic/superelastic memory staple.

18. The medical device of claim 17, wherein the medical staple is a nitinol staple.

19. The medical device of claim 15, wherein the medical device is reusable.

20. The medical device of claim 15, wherein the medical device is disposable.
